(19) United States
(12) Patent Application Publication
(10) Pub. No.: US 2008/0082020 A1
(43) Pub. Date: Apr. 3, 2008

(54) System and method for biofeedback administration

(76) Inventor: Thomas F. Collura, Chagrin Falls, OH (US)

Correspondence Address: ROETZEL & ANDRESS, 1375 EAST 9TH STREET, CLEVELAND, OH, 44114, US

(21) Appl. No.: 11/512,949

(22) Filed: Aug. 30, 2006

(51) Int. Cl. A61B 5/04

(52) U.S. Cl. 600/545

(57) ABSTRACT

Various embodiments of a biofeedback system, programs and methods are provided. In one example embodiment, a biofeedback system for administration of electroencephalographic (EEG) neurofeedback training includes a plurality of electrodes sensors for placement on the head of a trainee and a switching head box comprising a plurality of contacts each of which connects to one electrode sensor. The system also includes an interface device which includes at least two EEG signal amplifiers and connects to the switching head box, and a computer comprising software for generating user-control functions which corresponds in real-time to EEG signals received by the interface device and processed by the computer. The switching head box includes a switch with at least two conductors and connects the electrode sensors to the interface device for transmitting EEG signals from the trainee to the computer.

### FIELD OF THE INVENTION

The invention pertains generally to EEG biofeedback for learning and controlling bio-electric characteristics of the brain which correspond to different mind states. More particularly, the invention relates to system and method for obtaining quantitative EEG measurements and values from sensors positioned at various locations of the brain.

### BACKGROUND

Biofeedback is the recording, monitoring and analyzing of electrical activity of the brain and a corresponding metal state of a user. A plurality of visual, auditory and/or tactile feedback mechanisms are (integrated) with the electrical activity of the brain to facilitate neurofeedback training of the user. The interface is provided in such a manner so as to provide the ability of the user, in the case of self-administered monitoring, or the trainer, in the case of an administered session, to record, manage and control brain activity for different purposes including self-improvement.

EEG (brainwave) signals have been extensively studied in an effort to determine relationships between frequencies of electrical activity or neural discharge patterns of the brain and corresponding mental, emotional or cognitive states. Biofeedback of identified frequency bands of EEG signals is used to enable a person to voluntarily reach or maintain a target mental state. Frequency bands of EEG readings used in such biofeedback have been generally categorized in the approximate frequency ranges of: delta waves, 0 to 4 Hz; theta waves, 4 to 7 Hz; alpha waves, 8 to 12 Hz; beta waves, 12 Hz to 36 Hz, and sensorimotor rhythm (SMR) waves, 12 to 15 Hz.

It is theorized that each of the major subbands of biofeedback EEG (delta, theta, alpha, beta) has unique bio-electric characteristics which correspond with unique subjective characteristics of an individual. The delta band is observed most clearly in coma and deep sleep, the theta band in light sleep and drowsiness, the alpha band in a variety of wakeful states involving creativity, calm and inner awareness, and the beta band in alert wakeful situations with external focus. In general, a dominant brain wave frequency increases with increasing mental activity.

Many different approaches have been taken to EEG biofeedback to achieve mental state control. For example, U.S. Pat. No. 4,928,704 describes a biofeedback method and system for training a person to develop useful degrees of voluntary control of EEG activity. EEG sensors are attached to cortical sites on the head for sensing EEG signals in a controlled environment. The signals are amplified and filtered in accordance with strict criteria for processing within time constraints matching natural neurologic activity. The signals are filtered in the pre-defined subbands of alpha, theta, beta and delta, and fed back to the monitored person in the form of optical, aural or tactile stimuli.

QEEG devices typically records a minimum of 19-20 channels, for data acquisition and analysis to map brain activity. These devices have individual EEG signal amplifiers for each channel and are expensive and complicated systems to run, requiring an expert in the field to conduct training. Currently, substantially less expensive systems which have a lower number of channels, for example, two to four channel devices, which includes an amplifier for each channel can also be used. However, in a two-channel interface device, for example, the trainee or trainer is required to take additional time to reposition the conductors to two different sites on the head for each recording. Thus, in many of the conventional EEG biofeedback systems and methods, it is necessary to interrupt data collection to reposition the conductors, and in some cases, to also perform set-up functions, review component values, or set protocols or adjust threshold levels. These functions are typically performed by a session administrator, which can ultimately diminish or otherwise adversely effect the nature and quality of biofeedback signals to a trainee seeking to benefit from EEG training.

### SUMMARY

The present invention provides for a system, program and method of recording brainwaves around the head quickly and cost effectively on a low number of channels relative to a QEEG system. It provides recording from a relatively low number of channels to multiple sensor locations, and also provides a system and method to switch between channels instantly to obtain quality biofeedback.

In one embodiment, the present invention provides for a system for administration of electroencephalographic (EEG) neurofeedback training which includes a plurality of electrode sensors for placement on the head of a trainee, a switching head box electrically connected to the at least two sensors, an interface device which includes at least two EEG signal amplifiers and is electrically connected to the switching head box, and a computer electrically connected to the interface device and which includes software for generating user-control functions which correspond in real time to EEG signals received by the interface device. The switching head box includes a switch having a first conductor at a first position which connects a first electrode sensor to a first EEG signal amplifier of the interface device, and a second conductor at a second position which connects a second electrode sensor to a second EEG signal amplifier, for transmitting EEG signals from the trainee to the computer.

In another embodiment of the invention, a program embodied in a computer readable medium includes logic that simultaneously identifies at least two independent EEG brainwave signals received by at least two electrical sensors placed on a head of a trainee undergoing biofeedback training. The program includes logic which executes processing of the EEG brainwave signals and records EEG brainwave data derived from the EEG brainwave signals and logic that detects a predetermined time setting for processing the EEG brainwave signals and executes a prompt, at the conclusion of the predetermined time setting, to advance a switch if additional electrical sensors are to be processed.

### BRIEF DESCRIPTION OF THE SEVERAL VIEWS OF THE DRAWINGS

The various embodiments of the present invention can be understood with reference to the following drawings. The components in the drawings are not necessarily to scale. Also, in the drawings, like reference numerals designate corresponding parts throughout the several views.

FIG. 1 is a block diagram of the hardware components of a biofeedback system according to an embodiment of the invention;

FIG. 2 is a schematic diagram of the biofeedback system of FIG. 1, according to an embodiment of the invention;

FIG. 3 is an electrical schematic diagram of a two-channel, six-position switching head box of the biofeedback system of FIG. 1, according to an embodiment of the invention;

FIG. 4 is an electrical schematic diagram of a four-channel, 5-position switching head box of the biofeedback system of FIG. 1, according to an embodiment of the invention;

FIG. 5 is an electrical schematic diagram of a 2-channel, 2-position switching head box of the biofeedback system of FIG. 1, according to an embodiment of the invention;

FIG. 6 is a flow chart that provides an example of the logic that is executed in the controller of an interface device of the biofeedback system of FIG. 1, according to an embodiment of the invention; and

FIG. 7 is a screen display generated by monitoring logic of the biofeedback system of FIGS. 1 and 2, according to an embodiment of the invention.

### DESCRIPTION OF EMBODIMENTS

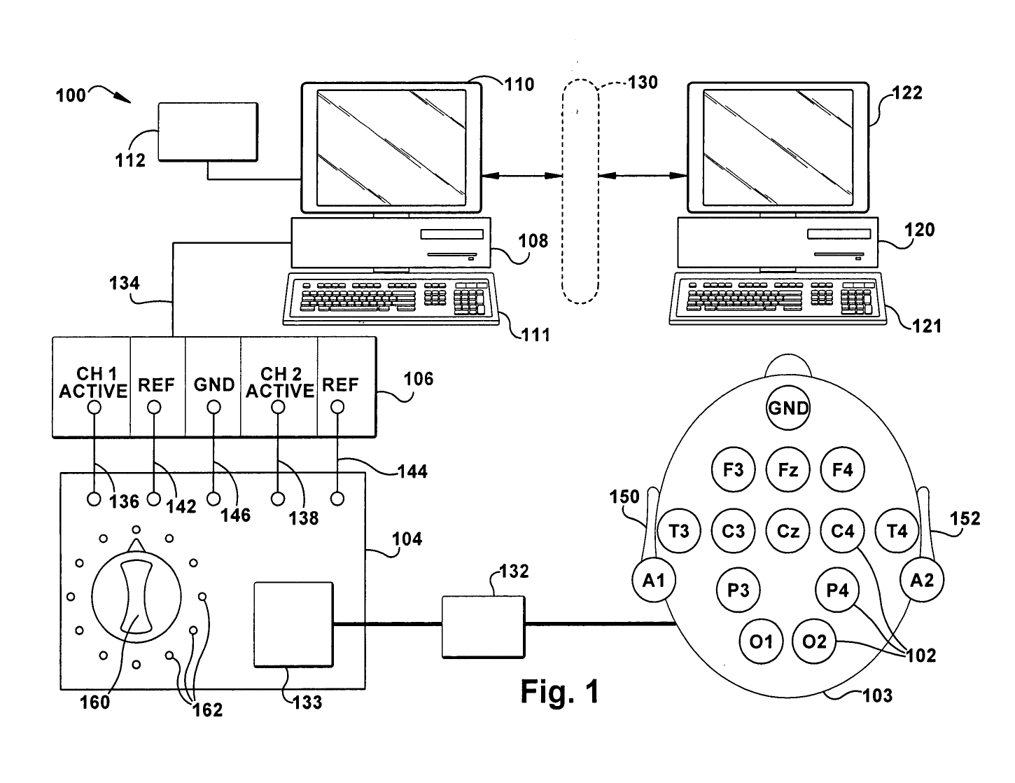

FIG. 1 is a block diagram of the hardware components of a biofeedback system 100 according to an embodiment of the invention. The biofeedback system 100 includes a plurality of electrodes 102 attachable to an electro-cap that is placed on the head 103 of a subject or trainee undergoing biofeedback training. The biofeedback system 100 further includes a switching head box 104, a user interface device 106, and a trainee computer or data processor 108 which is electrically connected to a display monitor 110, keyboard 111, and optionally, additional biofeedback stimulative devices 112 such as audio or vibratory headphones, light goggles, and/or tactile stimulator. These devices may be controlled by a feedback device controller (not shown) connected to user computer 108. The user computer 108 contains EEG analysis and biofeedback software which performs EEG recording, analysis and biofeedback operations, as will be further described herein. The biofeedback system can optionally include a trainer computer 120 having keyboard 121 and display monitor 122, in which the trainer computer 120 is connected to the trainee computer 108 either as another computer in a networked environment or at a remote location via the internet 130.

The EEG signals from the trainee undergoing biofeedback training flow from electrodes which connect to the switching head box 104 via a pigtail connector 132 or individually to individual pin-type connections (not shown) to connector 133 on the switching head box 104. The interface device 106 electrically connects to the trainee computer 108 via cable connector 134 and interface device 106 electrically connects to the switching head box 104 through various serial data lines, for example line 134 to channel 1 (CH1), line 138 to channel 2 (CH 2), lines 142 and 144 to reference and line 146 to ground. The switching head box 104 includes a selector switch 160 that can be turned to a plurality of positions 162. The selector switch 160 allows the trainee or trainer to easily select the electrodes for data collection and to control the reading of various areas of the head that are transmitting EEG data to the trainee computer 108. Thus the selector switch 160 prevents the trainee or trainer from having to move the electrodes to various positions on the head in order to obtain several EEG readings. The trainee can use a standard EEG cap and can easily select various areas of the brain in a short time. Furthermore, the software within the trainee computer 108 can prompt the trainee or trainer to switch the channels at a pre-determined time period to collect data a several electrodes to complete a biofeedback training session, as will be further discussed. Therefore, switching head box 104 allows the trainee or trainer to select which electrodes will be transmitted through to the interface device 106 and sent to the trainee computer to be read by the software therein. The interface device 106 reads the EEG signals coming into lines 136 and 138 converts them to digital form, and sends the digital signals to the computer 108 and the signals can then be viewed and interpreted on software, for example, Windows Operating System.

FIG. 1 also shows location of the plurality of electrodes 102 attached to the trainee head 103 as, for example a neutral (or “indifferent”) electrode to each ear, electrodes A1 and A2, and at least one electrode to locations on the scalp, for example, one on each side of the forehead C3 and C4 to provide “right active” and “left active” two-channel input, and a “ground” GND electrode. Generally, the active electrode will be attached to the head in a specific location (frontal, parietal, occipital, etc.), and the indifferent and ground electrodes will be attached to each ear. The active and indifferent electrodes connect through the switching box 104 and then to the interface device 106. For example, when the selector switch 160 is turned to a single position of the plurality of switch positions 162, and with the active electrodes C3 and C4 attached to the head 103, the indifferent electrodes A1 and A2 attached to the left and right ears, the switching head box 104 and the interface device will track (measure) brainwave activity between the head and the left and right ears as references, and sensor GND on forehead used as ground. Therefore, in one example embodiment, two active leads C3 and C4 can provide EEG monitoring through channel 1, CH 1, and channel 2, CH 2, respectively, of the interface device 106.

In addition, several additional active leads may connect to channels 1 and 2, respectively. For example, when the selector switch 160 is turned to a single position, of the plurality of switch positions 162, active electrodes C3, C4 can provide monitoring through channel 1 and electrodes P3 and P4 can provide monitoring to channel 2. Selector switch 160 may then be turned to a new position and active electrodes T3, T4 can provide monitoring through channel 1 and electrodes O1, O2 can provide signals through channel 2. Therefore two or more electrode connections can be read in channel 1 while two or more electrode connections can be read in channel 2. The selector switch 160 can then be turned so that additional electrodes may be read via channels 1 and 2. In an alternative embodiment, the switching head box 104 can have additional channels, for example 10 or more channels.

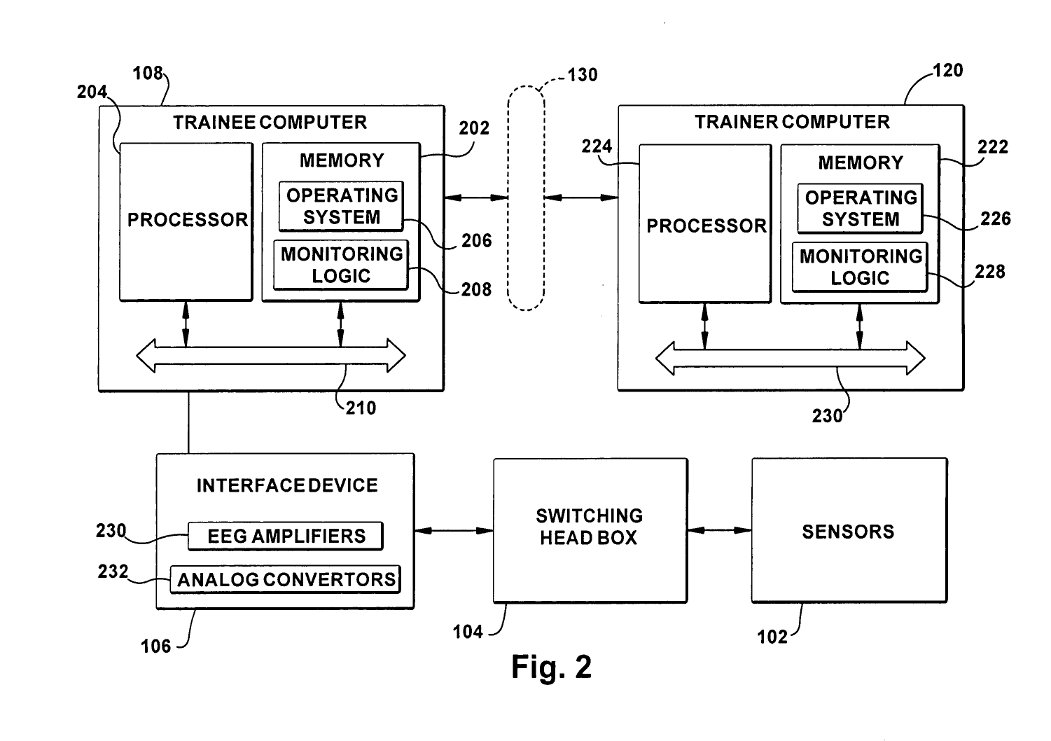

FIG. 2 is a schematic diagram of the biofeedback system of FIG. 1 which includes the sensors 102, switching head box 104, interface device 106, trainee and trainer computers 108, 120 all of which are electrically coupled to one another. The example embodiment of FIG. 2 is described with reference to a trainee computer 108 that is directly coupled to interface device 106 which selectively reads EEG signals via sensors 102 on trainee head through switching head box 104. The trainee computer 108 could be directly coupled to trainer computer 120, or alternatively, the trainee computer 108 could interface with a trainer computer 120 in a networked environment or via the Internet, intranets, wide area networks (WANs), local area networks, wireless networks, or other suitable networks, etc., or any combination of two or more such networks. The trainee and trainer computers 108, 120 may be, for example, desktops, laptops, palm or hand held computers such as a personal digital assistant, or any other devices with like capability.

The trainee computer 108 includes software or firmware components that are stored in the memory 202 and are executed by the processor 204, and each are coupled to respective local interface 210, for example an input/output data bus which can also connect to keyboard 111 and biofeedback stimulative devices 112 (FIG. 1). The trainer computer 120, if present, also includes software or firmware components that are stored in the memory 222 and are executable by the processor 224, and are coupled to local interface 230. These components include, for example, operating systems 206, 226 and monitoring logic 208, 228. The operating systems 206, 226 are executed to control the allocation and usage of hardware resources such as the memory, processing time and peripheral devices 111, 112, 121 (FIG. 1). In this manner, the operating systems 240, 250 serve as the foundation on which applications depend. Monitoring logic 208, 228 monitors trainee EEG signals and provides feedback for biofeedback training. For example, the monitoring logic 208 of trainee computer 108 may include logic that performs EEG signal processing for EEG frequency band measurement and to generate images of these brainwave measurements, logic that makes a determination of the information via computation functions, logic that carries out a number of possible user feedback tasks which can be displayed on trainee monitor 110 (FIG. 1), logic that sorts, saves and restores data files, and logic which provides summary reporting and graphing capabilities.

As used herein, the term “executable” means a program file that is in a form that can ultimately be run by the processors 204, 224. Examples of executable programs may be, for example, a compiled program that can be translated into machine code in a format that can be loaded into a random access portion of the memories 202, 222 and run by the processors 204, 224 or source code that may be expressed in proper format such as object code that is capable of being loaded into a random access portion of the memories 202, 222 and executed by the processors 204, 224 etc. An executable program may be stored in any portion or component of the memories 202, 222 including, for example, random access memory, read-only memory, a hard drive, compact disk (CD), floppy disk, or other memory components.

The memories 202, 222 are each defined herein as both volatile and nonvolatile memory and data storage components. Also, each of the processors 204, 224 may represent multiple processors and each of the memories 202, 222 may represent multiple memories that operate in parallel processing circuits, respectively. In such a case, each of the local interfaces 210, 230 may be an appropriate network that facilitates communication between any two of the multiple processors, between any processor and any of the memories, or between any two of the memories, etc.

The interface device 106 acquires and transmits data, and the trainee computer 108 receives and processes the data to make a determination of the information, and then carries out any of a number of possible user-feedback tasks which can be displayed the display monitor 110 connected to the user computer 108. As mentioned above, interface device 106 receives data from sensors 102 via switching head box 104 through the data serial lines 136, 138 (FIG. 1) which it transmits to the trainee computer 108. Interface device 106 includes two or more EEG signal amplifiers 230, one for each channel of data transmission. As shown in FIG. 1, the interface device 106 is transmitting 2 channels of data and therefore has two EEG signal amplifiers 230, although additional channels of data are possible, for example 2-10 channels, in another example, 2-8 channels, 2-6 channels, 2-4 channels and all combination of numbers of channels therebetween. Interface device 106 includes firmware in the way of analog converters 232 which read the incoming analog EEG signals from electrode sensors 102, converts them to digital form, and sends the digital signals to the trainee computer 108. The digital signals can then be viewed and interpreted on software installed on the trainee computer 108, as will be further described.

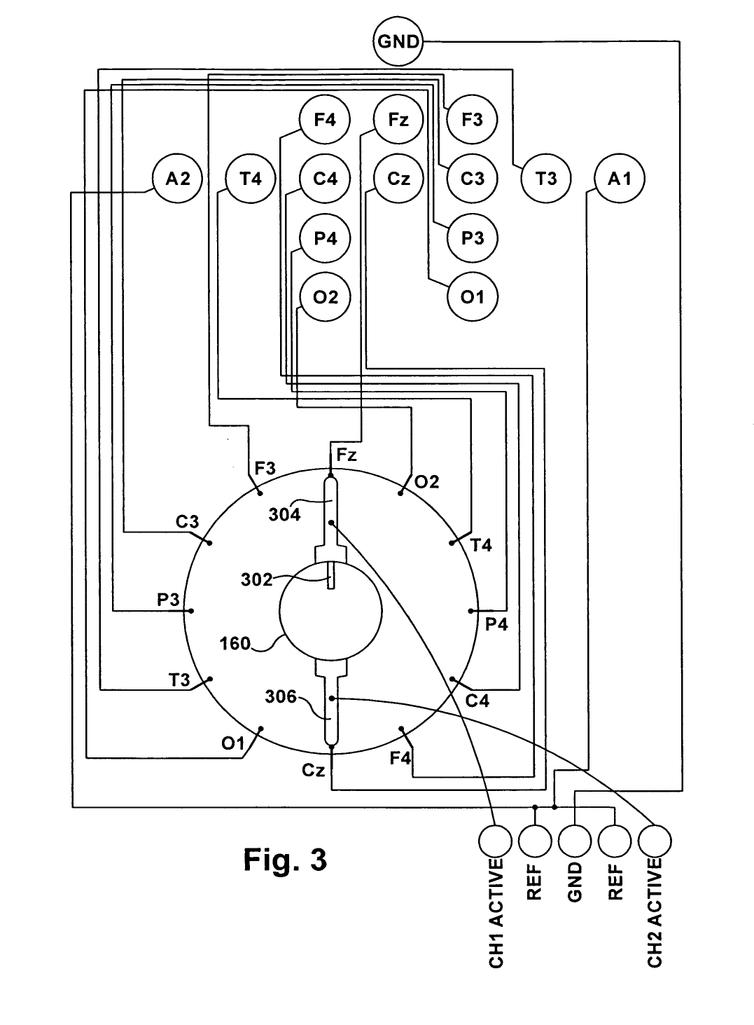

Next, a general description of the operation and functioning of switching head box 104 is provided within the context of the biofeedback system 100 of FIGS. 1 and 2. FIGS. 3 through 5 show example electrical schematics of switching head box 104 configured to receive data from the electrode sensors 102 (FIGS. 1 and 2) and to transmit the data to the interface device 106 (FIGS. 1 and 2) through two or more channels. Each electrical schematic illustrates one of several possible electrical circuits are established via the switch 160 at the various switch positions that may be selected. As illustrated, the switch 160 has two conductors 304, 306 which make contact with the electrode sensors 102 and two active channel ports, CH1, CH2. Each conductor 304, 306 connects to one electrode sensor 102, and so, switch 160, as shown, connects to two electrode sensors on the head of the trainee undergoing biofeedback treatment when the switch is located at each switch position 160. As stated above and as shown in the example embodiments described below, each channel of the switching head box 104 interfaces with a separate amplifier of the interface device 106 through channel ports CH1, CH2.

In the example embodiment shown in FIG. 3, data is transmitted from a first electrode sensor FZ located on the head of the trainee undergoing feedback treatment to contact FZ of switching head box 104, and through conductor 304 of switch 160 which electrically connects to Channel 1 port, CH1, to interface device 106. At the same time, data from a second electrode sensor, CZ located on the head of the trainee is transmitted through conductor 306 of switch 160 and to Channel port two, CH2 of switching head box 104 to interface device 104. After the data is passed through separate EEG signal amplifiers of interface device 106, the data is transmitted to trainee computer 108 (FIG. 1). Therefore, activation of switch 160 to a first position as indicated by position indicator 302 completes two electrical circuits that allows current to pass through two separate electrode sensor sites of the brain to the interface device 106 and to the trainee computer 108. In the embodiment shown, the switch 160 can be turned to six positions in which the conductors make contact with all twelve electrode sensor sites. A suitable switch can be any switch, for example, a double-pole switch that can move to two or more positions and that is capable of completing at least two electrical circuits that connect two electrode sensors to two distinct channel ports, CH 1 and CH 2, of switching head box 104 and to interface device 106 and to two distinct EEG signal amplifiers, of interface device 104. The switching head box 104 of FIG. 3 is designed such that switch 160 has two conductors that interface with 12 electrode sites and where the switch 160 can be moved to six positions to read data to electrode sensor sites at each switch position. Accordingly, when switch 160 is placed in a second, third, fourth, fifth and sixth electrical contacts at six positions, electrical contact is made and therefore data can be read from electrode sensor pairs C3 and C4, P3 and P4, T3 and T4 and O1 and O1, respectively.

In alternative embodiments, switching head box 106 can be configured to receive data from a large range of electrode sensor sites. For example, the number of sensor sites that can be read depend on the number of electrode sites or the electrode cap that is placed on the head of the trainee and can range anywhere from 2-256 sites and another example can range from 2-64, and another embodiment from about 2-32 and in another embodiment from about 2-20, and in still yet in another embodiment from about 2-12 electrodes and all ranges therebetween. In addition, switch 160 of switching head box 106 can include at least two conductors, depending upon the number of channel ports and channels that can be read by interface device 106.

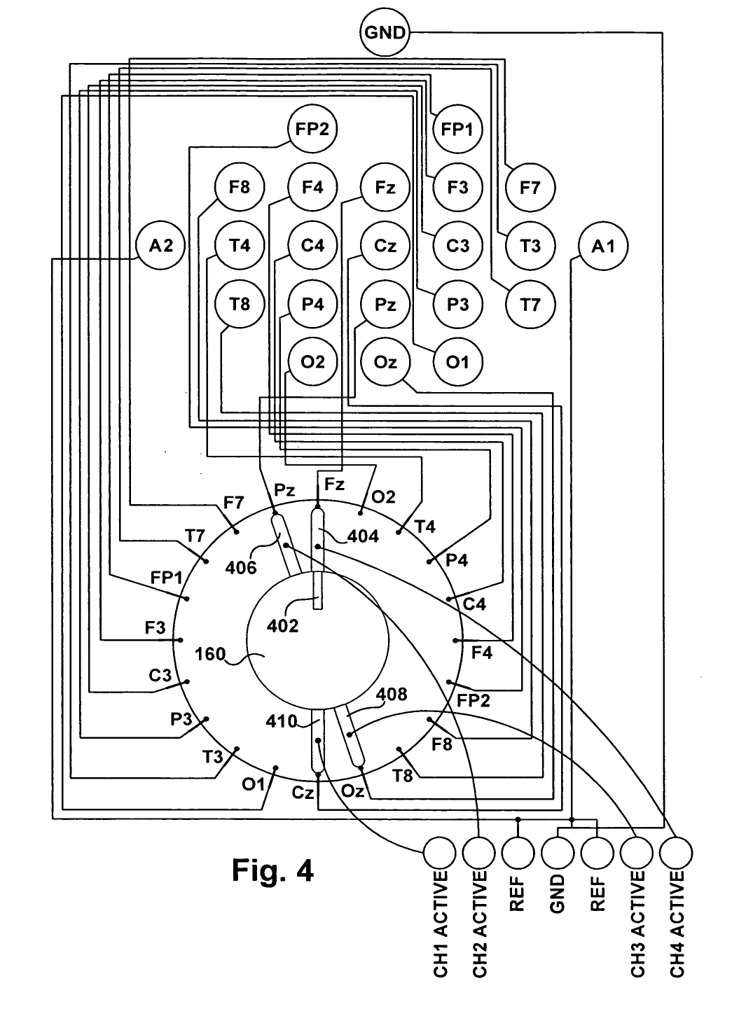

FIG. 4 illustrates an electrical schematic of a switching head box 104 that reads data from 20 electrode sensor sites. In addition, switching head box 106 includes four distinct channel ports, CH1, CH2, CH3 and CH4, which can allow for the transmission of data for four separate EEG signal amplifiers of interface device 106. Switch 160 has four conductors, 404, 406, 408, 410, which make contact with four contacts at four positions to read four distinct electrode sensors. Switch 160 can be rotated to five different positions in order to transmit the data from all twenty electrode sensor sites. Accordingly, switching head box 104 of FIG. 4 is a four channel, 5 position switching head box 104. When switch 160 is placed in a first position, as indicated by position indicator 402, contact 404 makes contact with electrode site FZ, contact 406 makes contact with electrode sensor site PZ, electrode 408 makes contact with electrode sensor site OZ and conductor 410 makes contact with electrode sensor site CZ. As shown, data from electrode sensor site CZ is transmitted to the Channel 1 port, CH1, the data from electrode sensor site PZ is transmitted to Channel port 2, CH 2, the data from electrode sensor site OZ is transmitted to Channel 3 port, CH 3, and the data from electrode sensor site FZ is transmitted to Channel port 4, CH4. Thus, four separate circuits can be established simultaneously through switch 160 of switching head box 106. Movement of switch 160 to a second position breaks the circuit to electrode sites Cz, Pz, Oz and Fz and establishes connection to four new sites, for example, T4, P4, P3, and T3. Since, in the embodiment of FIG. 4, the number of electrode sites is 20, the switch can be placed in a third, fourth and fifth position to make electrical contact with electrode sites P4, C4, P3 and C3; and F4, FP, 2, F3, and FP1; and F8, T8, T7 and F7, respectively.

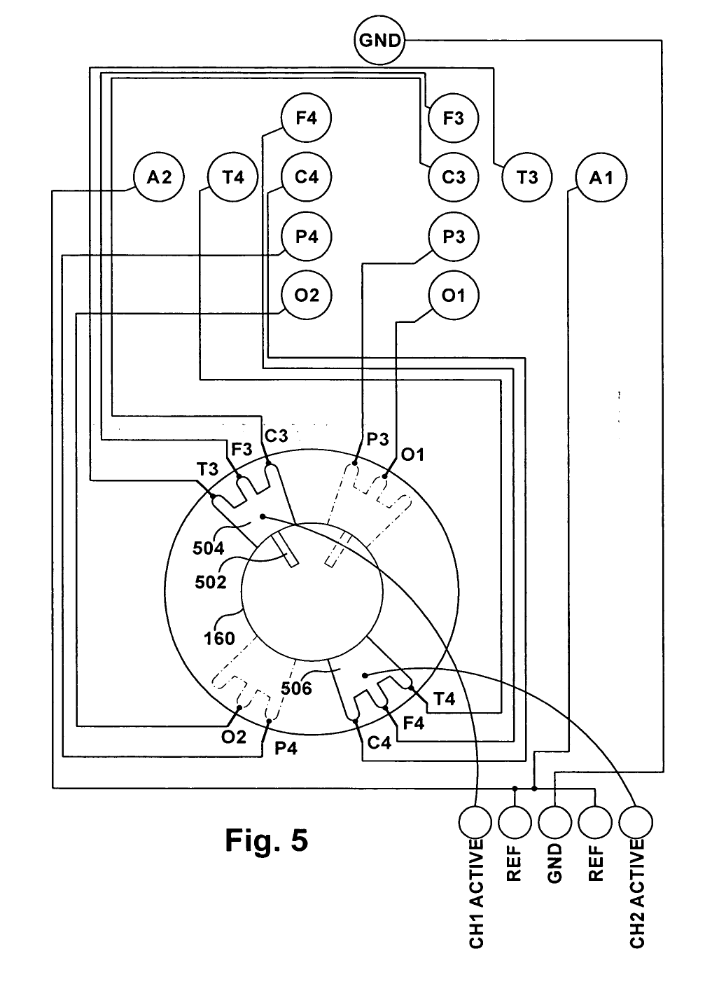

It should be understood, that any four sensors can be chosen for connection at a given time. For example, although electrode sensors Fz, Pz, Oz are shown to make connection at the same time, other alternative sites can be made by conductors 404, 406, 408 and 410. Thus, in the example embodiments of FIGS. 4 and 5, each conductor makes contact with one electrode sensor site and transmits data to a single EEG signal amplifier. In addition, it is also possible that switch 160 of FIG. 4 includes two conductors, for example, or any number of conductors greater than two.

In conducting biofeedback training, it may be desirable to train whole sections of the brain. The biofeedback system 100 can also conduct training based on combined signals to perform a computation of coherence which is known as “synchrony training”. FIG. 5 shows a switch 160 having at least two conductors which receives data from at least two electrode sensor sites, at each electrode. For example, switch 160 is at a first position as indicated by indicator position 502, and contact is made to electrode sensors F3, T3 and C3 which are connected in parallel to provide a first channel reading to channel 1 port, CH1. Contact is also made via contact 506 to electrode sensors F4, T4 and C4 which are connected in parallel to provide a second channel reading which is transmitted to channel 2 port, CH 2. Therefore, each of the conductors 504 and 506 of switch 160 make connection to more than one sensor which transmits to each channel, and so data from the several electrode sensors are provided with only two EEG signal amplifiers. This electrical arrangement in conjunction with computation performed by the logic provides an average reading of the electrical activity of at least two electrode sensor sites. This method may be referred to as “volume-conduction averaging” and is a method for training multiple brain sites. This allows for sychrony training that is sensitive to the amplitude and phase synchrony of the different sites. Switch 160 can then be moved clockwise so that the position indicator 502 aligns with the second position and conductor 504 makes contact with electrode sensors P3, O1 and conductor 506 makes contact with P4 and O2, which transmits signals to Channel 1 port, CH1 and Channel 2 port, CH2, respectively.

The specific combinations of sensors is a matter of design choice and can be variable. That is, the specific numbers or pairs or quads, etc., and combinations of sensors employed depend upon the desired training. Homologous pairs can be chosen such that contact 504 connects to all sensors on the left side of the brain, for example electrode sensors F3, T3, C3, P3, O1, and conductor 506 connects all sensors on the right side of the brain, for example, electrode sensor sites F4, T4, C4, P4, O2. Therefore synchrony training can conduct the entire head training with 10 sites being read through CH1 and 10 sites being read through CH2. Again, it should be understood that the number of electrode sensors read can vary greatly and the number of conductors of switch 160 can be any number greater than two, each of which connects to a distinct channel amplifier of interface device 106.

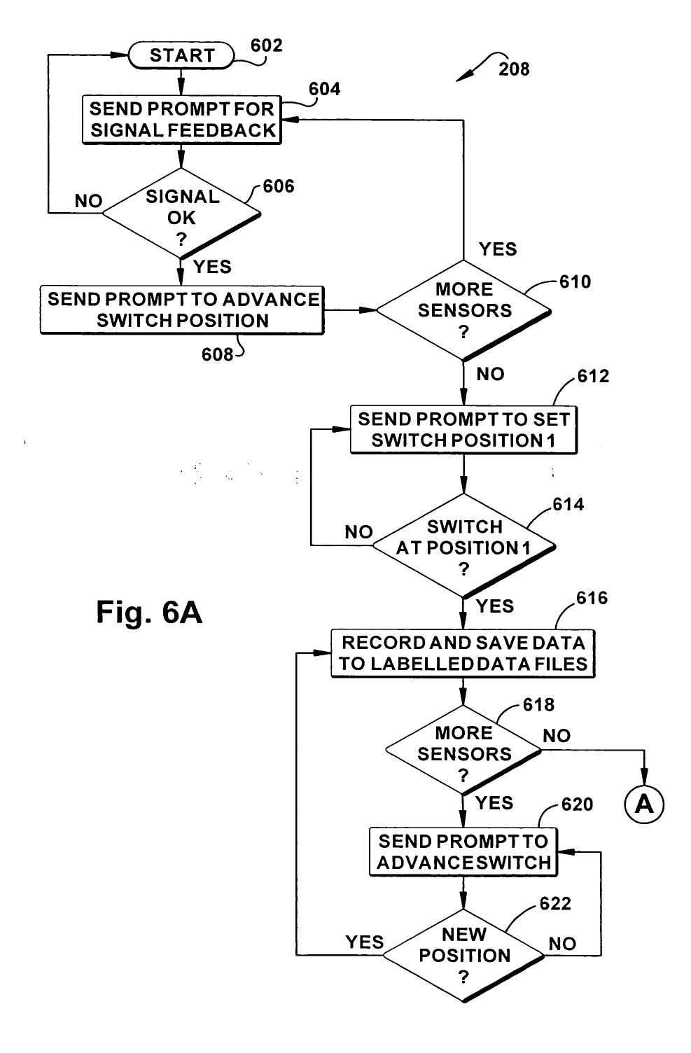

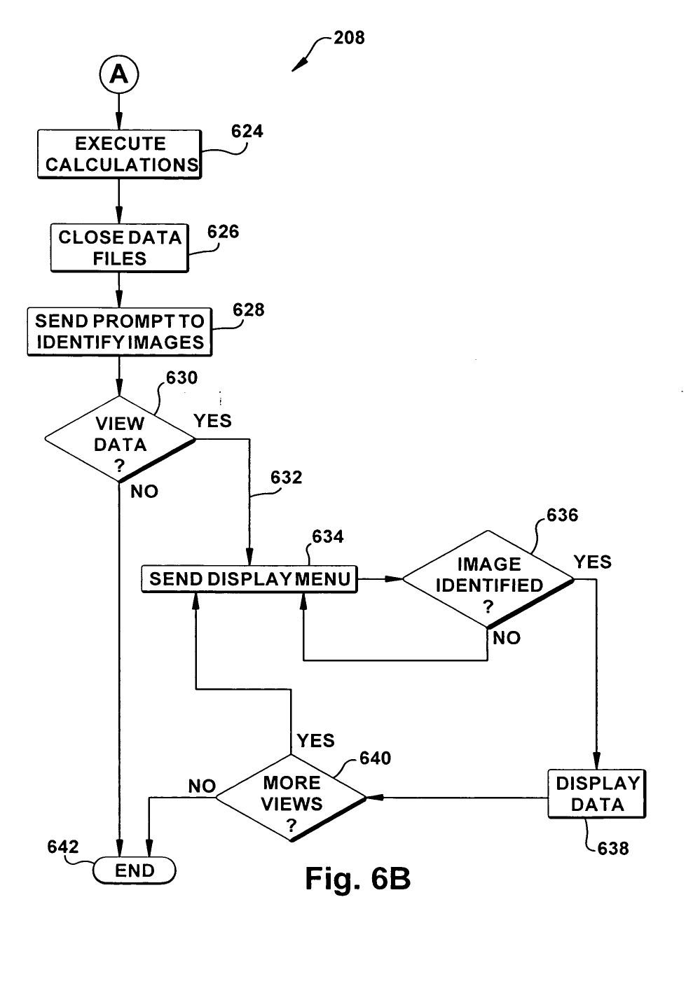

FIGS. 6A and 6B is a flow chart that provides an example embodiment of the monitoring logic 208 (FIG. 2) that is executed in a trainee computer, and optionally, a trainer computer of the biofeedback system of FIG. 1, according to an embodiment of the invention. FIGS. 6A and 6B show a flow chart of one example of the monitoring logic 208 according to an embodiment of the present invention. Alternatively, FIGS. 6A and 6B may be viewed as depicting steps of an example of a method implemented in a trainee computer 108 (FIG. 2) to determine the biofeedback readings of several sensors on the trainee's head. The functionality of the monitoring logic 208 as depicted by the example flow chart of FIGS. 6A and B may be implemented, for example, in an object-oriented design or in some other suitable programming architecture. Assuming the functionality is implemented in an object-oriented design, each block represents functionality that may be implemented in one or more methods that are encapsulated in one or more objects. The monitoring logic may be implemented using any one of a number of programming languages such as, for example, C, C++, JAVA, Perl, or other suitable programming languages.

Beginning with box 602, the monitoring logic 208 sends a prompt at box 604 to the user, for example via display monitor 110 of computer 108, and the logic at box 606 determines whether or not the signal is sufficiently strong. Assuming that the signal is good, then at box 608 a prompt is sent to advance the switch position. The monitoring logic 208 then determines at box 610 whether or not there are any more signals from electrode sensors to be read for data. If the response is “Yes” then another prompt is sent for signal feedback at box 604 and to determine whether the signals from additional electrode sensors are sufficiently strong at box 606. If all of the signals are not sufficiently strong, then the monitoring logic starts over at 602.

Once there are no more electrode sensors to be read, then in box 612 the monitoring logic 208 sends a signal to prompt the user to set the switch position to the first switch position. The monitoring logic 208 then determines whether or not the switch has been advanced to the first position in box 614. If the switch position has not been set to position 1, the prompt will continue to be sent to the monitor 110 of the trainee computer 108. Once the switch position is set to position 1, the monitoring logic then records and saves data at box 616 to labeled data files within the memory 202 of trainee computer 108. Once that data is recorded and saved, the monitoring logic 208 determines whether there are any additional sensors to be read at box 618.

Assuming there are more sensors to be read, then the monitoring logic 208 sends a prompt to advance the switch at box 620. The monitoring logic then determines, at box 622, whether or not the switch has been advanced to a second position. Once the switch has been set to a second position, then the monitoring logic 208 records and saves the data to the labeled data files at box 616. This process starting at box 616 is repeated until all of the sensors have been read and the data have been saved and labeled to the data files. Once all of the data from all of the sensors have been read, then at box 624 the monitoring logic executes calculations and interpretations on the data. Once all the calculations have been executed, then the monitoring logic closes the data files at box 626 and then a prompt is sent to the user to identify images at box 628.

Next, the user can determine whether or not he or she wants to view the data that is being stored and labeled at box 630 where a prompt is sent to request action on the part of the user as to whether or not they want to view the data. If there is no interest in viewing the data, then the user can indicate “No” and the program will end. However, if the trainee and user wishes to view the data, then monitoring logic 208 sends a display menu at box 634, for example to the monitor 110 of the trainee computer 108. The logic then asks whether or not a particular image to be viewed has been identified by the user or trainee at box 636. If a choice of image has not been identified, then the monitoring logic will maintain the display prompt. However, once the trainee or user indicates a choice of the image to be identified from the display menu, then the monitoring logic at box 638 will display the data. Once the data has been displayed the monitoring logic provides the choice as to whether or not the trainee or user would like to see additional views of the data at box 640. Once the user has responded to the prompt “Yes” to see additional display menus, then the logic determines whether another image has been identified from the display menu in response to the prompt. Once a response to the prompt has been made by the user or trainee, then additional data can be displayed. The monitoring logic 208 will continue to prompt the user until the user responds to the prompt with a “No”, in which case the program will end at 642.

Thus, in one example embodiment of the invention, the monitoring logic 208 is configured such that it will continue to read all of the sensors and once the sensors have been read, prompts will be sent to change the switch position until the user or trainee no longer advances the switch positions. If the user responds that there are no more sensors to be read, then the monitoring logic continues into the calculation mode and display mode, in which case the user has several choices by which it can view images of the data and the calculations performed on the data.

Although the flow chart of FIGS. 6A and B shows a specific order of execution, it is understood that the order of execution may differ from that which is depicted. For example, the order of execution of two or more blocks may be changed relative to the order shown. Also two or more blocks shown in succession in FIGS. 6A and B may be executed concurrently or with partial concurrence. In addition, any member of counters, state variables, warning semaphores, or messages might be added to the logical flow described here, for purposes of enhanced utility, accounting, performance measurement, or providing trouble shooting aids, etc. It is understood that all such variations are within the scope of the present invention.

Although the monitoring logic 208 is embodied in software or code executed by general purpose hardware as discussed above, as an alternative each may also be embodied in dedicated hardware or a combination of software/general purpose hardware and dedicated hardware. If embodied in dedicated hardware, the monitoring logic 208 can be implemented as a circuit or state machine that employs any one of or a combination of a number of technologies. These technologies may include, but are not limited to, discrete logic circuits having logic gates for implementing various logic functions upon an application of one ore more data signals, application specific integrated circuits having appropriate logic gates, programmable gate arrays (PGA), field programmable gate arrays (FPGA), or other components, etc. Such technologies are generally well known by those skilled in the art and, consequently, are not described in detail herein.

Also, where the monitoring logic 208 comprise software or code, each can be embodied in any computer-readable medium for use by or in connection with an instruction execution system such as, for example, a processor in a computer system or other system. In the context of the present invention, a “computer-readable medium” can be any medium that can contain, store, or maintain the monitoring logic 208 for use by or in connection with the instruction execution system. The computer readable medium can comprise any one of many physical media such as, for example, electronic, magnetic, optical, electromagnetic, infrared, or semiconductor media. More specific examples of a suitable computer-readable medium would include, but are not limited to, magnetic tapes, magnetic floppy diskettes, magnetic hard drives, or compact discs. Also, the computer-readable medium may be a random access memory (RAM) including, for example, static random access memory (SRAM) and dynamic random access memory (DRAM), or magnetic random access memory (MRAM). In addition, the computer-readable medium may be a read-only memory (ROM), a programmable read-only memory (PROM), an erasable programmable read-only memory (EPROM), an electrically erasable programmable read-only memory (EEPROM), or other type of memory device.

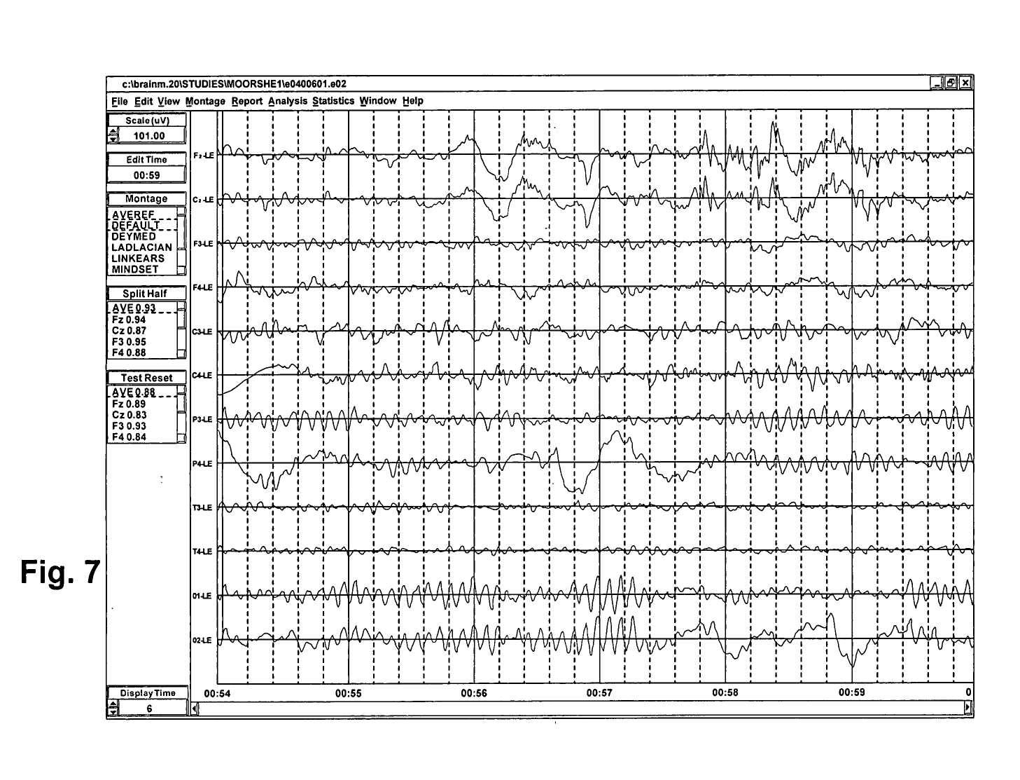

FIG. 7 illustrates an example of an EEG wave form signal display of a scrolling raw wave form using one configuration of the biofeedback system of the present invention the wave form displays a test protocol, for example, which records a series of six, one second epochs of therefore displaying one second of EEG monitoring at each of (how many? 12?) electrode sensors at (six?) different switch positions. The data can be obtained without disturbing the neurofeedback training session. A trainee can use a standard EEG electrocap having a plurality of electrode sensor positions. Also, the length of time can vary at each electrode sensor position, for example to one minute intervals for each of the six positions, thereby completing the analysis in six minutes. This capability allows for the application of self-administered biofeedback training which eliminates the need for a dedicated operator or session administrator to monitor waveforms, independent of the trainee's activity.

Table I displays the EEG data derived from the EEG signals, for example, a textual summary of the EEG component values, their means, and standard deviations, for predetermined time intervals, or whenever prompt to a response is made.

TABLE I

| | | | | | | | | | | | | TH/ | | TH/ | |
|---|---|---|---|---|---|---|---|---|---|---|---|---|---|---|---|
| RUN | NPTS | SITE | TYPE | DELTA | THETA | ALPHA | LOBET | BETA | HIBET | GAMMA | USER | AL | TH/LB | BE | AL/BE |
| 1 | 60 | Fz | MEAN | 10.46 | 8.38 | 9.51 | 6.31 | 12.08 | 9.95 | 15.19 | 4.71 | 0.88 | 1.33 | 0.69 | 0.79 |
| 1 | 60 | Fz | MEANF | 3.91 | 7.11 | 9.7 | 5.12 | 14.1 | 17.35 | 4.37 | 4.71 | 0.73 | 1.39 | 0.5 | 0.69 |
| 1 | 60 | Fz | STDDEV | 6.03 | 3.8 | 3.9 | 2.44 | 3.62 | 2.98 | 1.74 | 1.71 | 0.97 | 1.55 | 1.05 | 1.08 |
| 1 | 60 | Fz | MODFRQ | 1.47 | 5.29 | 9.84 | 13.4 | 17.59 | 24.27 | 39.73 | 32.24 | 0.54 | 0.39 | 0.3 | 0.56 |
| 1 | 60 | Cz | MEAN | 8.17 | 7.66 | 10.54 | 6.12 | 12.41 | 9.25 | 15.27 | 4.36 | 0.73 | 1.25 | 0.62 | 0.85 |
| 1 | 60 | Cz | MEANF | 3.13 | 6.61 | 11.29 | 5.88 | 15.19 | 16.5 | 4.47 | 4.28 | 0.59 | 1.12 | 0.44 | 0.74 |
| 1 | 60 | Cz | STDDEV | 2.85 | 3.1 | 5.55 | 2.55 | 4.12 | 2.45 | 1.67 | 1.41 | 0.56 | 1.22 | 0.75 | 1.35 |
| 1 | 60 | Cz | MODFRQ | 1.76 | 5.37 | 9.94 | 13.36 | 17.58 | 24.13 | 39.72 | 32.3 | 0.54 | 0.4 | 0.31 | 0.57 |
| 1 | 60 | Fz–Cz | COHE | 50.47 | 35.32 | 43.75 | 14.3 | 56.78 | 34.85 | 61.98 | 0 | 0.81 | 2.47 | 0.62 | 0.77 |
| 1 | 60 | Fz–Cz | PHASE | 16.32 | 15.97 | 12.42 | 14.5 | 9.03 | 12.55 | 0.15 | 7.67 | 1.29 | 1.1 | 1.77 | 1.38 |
| 1 | 60 | Fz/Cz | ASYM | 1.28 | 1.09 | 0.9 | 1.03 | 0.97 | 1.08 | 0.99 | 1.08 | 1.21 | 1.06 | 1.12 | 0.93 |
| 2 | 60 | F3 | MEAN | 7.87 | 7.22 | 7.28 | 5.94 | 12.26 | 14.21 | 12.75 | 5.77 | 0.99 | 1.22 | 0.59 | 0.59 |
| 2 | 60 | F3 | MEANF | 2.94 | 5.62 | 7.88 | 4.55 | 12.95 | 23.15 | 5.95 | 5.38 | 0.71 | 1.23 | 0.43 | 0.61 |
| 2 | 60 | F3 | STDDEV | 5.87 | 3.47 | 3.01 | 2.44 | 3.8 | 4.4 | 2.79 | 2.03 | 1.15 | 1.42 | 0.91 | 0.79 |
| 2 | 60 | F3 | MODFRQ | 1.5 | 5.29 | 10 | 13.42 | 17.7 | 24.22 | 39.89 | 32.42 | 0.53 | 0.39 | 0.3 | 0.56 |
| 2 | 60 | F4 | MEAN | 10.5 | 9.46 | 8.64 | 6.11 | 11.99 | 12.75 | 14.48 | 5.19 | 1.09 | 1.55 | 0.79 | 0.72 |
| 2 | 60 | F4 | MEANF | 3.75 | 7.48 | 9.71 | 4.8 | 12.67 | 19.73 | 4.52 | 5.14 | 0.77 | 1.56 | 0.59 | 0.77 |
| 2 | 60 | F4 | STDDEV | 6.73 | 4.37 | 3.65 | 2.4 | 4.1 | 3.82 | 1.98 | 1.92 | 1.2 | 1.82 | 1.06 | 0.89 |
| 2 | 60 | F4 | MODFRQ | 1.91 | 5.28 | 9.84 | 13.44 | 17.61 | 24.59 | 39.77 | 32.21 | 0.54 | 0.39 | 0.3 | 0.56 |
| 2 | 60 | F3–F4 | COHE | 47.78 | 38.45 | 39.2 | 12.58 | 47.35 | 35.68 | 36.12 | 0.53 | 0.98 | 3.06 | 0.81 | 0.83 |
| 2 | 60 | F3–F4 | PHASE | 16.87 | 16.07 | 14.52 | 21.13 | 18.2 | 26.65 | 4.18 | 29.12 | 1.11 | 0.76 | 0.88 | 0.8 |
| 2 | 60 | F3/F4 | ASYM | 0.75 | 0.76 | 0.84 | 0.97 | 1.02 | 1.12 | 0.88 | 1.11 | 0.91 | 0.78 | 0.75 | 0.82 |
| 3 | 60 | C3 | MEAN | 6.08 | 6.15 | 9.85 | 5.4 | 9.23 | 9.53 | 9.84 | 4.16 | 0.62 | 1.14 | 0.67 | 1.07 |
| 3 | 60 | C3 | MEANF | 2.94 | 5.8 | 11.8 | 6.07 | 13.79 | 20.22 | 4.72 | 5.13 | 0.49 | 0.96 | 0.42 | 0.86 |
| 3 | 60 | C3 | STDDEV | 2.98 | 2.61 | 4.66 | 2.1 | 3 | 3.4 | 1.9 | 1.45 | 0.56 | 1.25 | 0.87 | 1.55 |
| 3 | 60 | C3 | MODFRQ | 1.5 | 5.25 | 10.1 | 13.27 | 17.62 | 24.22 | 39.83 | 32.38 | 0.52 | 0.4 | 0.3 | 0.57 |
| 3 | 60 | C4 | MEAN | 6.7 | 6.85 | 9.24 | 5.43 | 9.63 | 7.98 | 12.59 | 3.35 | 0.74 | 1.26 | 0.71 | 0.96 |
| 3 | 60 | C4 | MEANF | 3.47 | 7.09 | 12.24 | 6.12 | 13.94 | 15.46 | 4.15 | 4.38 | 0.58 | 1.16 | 0.51 | 0.88 |
| 3 | 60 | C4 | STDDEV | 3.36 | 2.73 | 4.25 | 2.33 | 2.88 | 2.26 | 1.21 | 1.29 | 0.64 | 1.17 | 0.94 | 1.47 |
| 3 | 60 | C4 | MODFRQ | 1.86 | 5.27 | 9.94 | 13.3 | 17.51 | 24.38 | 39.65 | 32.19 | 0.53 | 0.4 | 0.3 | 0.57 |
| 3 | 60 | C3–C4 | COHE | 35.65 | 21.57 | 33.58 | 9.18 | 33.57 | 19.85 | 7.23 | 0.12 | 0.64 | 2.35 | 0.64 | 1 |
| 3 | 60 | C3–C4 | PHASE | 12.38 | 14.93 | 27.25 | 26.25 | 18.77 | 28.63 | 3.15 | 22.28 | 0.55 | 0.57 | 0.8 | 1.45 |
| 3 | 60 | C3/C4 | ASYM | 0.91 | 0.9 | 1.07 | 0.99 | 0.96 | 1.19 | 0.78 | 1.24 | 0.84 | 0.9 | 0.94 | 1.11 |
| 4 | 60 | P3 | MEAN | 5.55 | 5.55 | 11.81 | 5.52 | 7.39 | 7 | 7.49 | 3.07 | 0.47 | 1.01 | 0.75 | 1.6 |
| 4 | 60 | P3 | MEANF | 3.7 | 7.4 | 20.2 | 7.33 | 12.8 | 15.29 | 2.82 | 3.96 | 0.37 | 1.01 | 0.58 | 1.58 |
| 4 | 60 | P3 | STDDEV | 2.24 | 3.58 | 6.24 | 2.48 | 3.26 | 2.37 | 1.11 | 1.49 | 0.57 | 1.44 | 1.1 | 1.91 |
| 4 | 60 | P3 | MODFRQ | 1.54 | 5.31 | 10.07 | 13.2 | 17.46 | 24.15 | 39.62 | 32.26 | 0.53 | 0.4 | 0.3 | 0.58 |
| 4 | 60 | P4 | MEAN | 9 | 7.8 | 11.72 | 6 | 9.06 | 6.9 | 11.11 | 3.15 | 0.67 | 1.3 | 0.86 | 1.29 |
| 4 | 60 | P4 | MEANF | 4.12 | 8.12 | 16.58 | 6.47 | 13.08 | 13.32 | 3.43 | 3.37 | 0.49 | 1.26 | 0.62 | 1.27 |
| 4 | 60 | P4 | STDDEV | 2.9 | 4.71 | 6.13 | 2.56 | 3.38 | 1.91 | 1.03 | 1.13 | 0.77 | 1.84 | 1.39 | 1.81 |
| 4 | 60 | P4 | MODFRQ | 1.78 | 5.34 | 9.94 | 13.25 | 17.46 | 24.21 | 39.64 | 32.14 | 0.54 | 0.4 | 0.31 | 0.57 |
| 4 | 60 | P3–P4 | COHE | 40.35 | 25.08 | 47.15 | 10.08 | 26.97 | 12.42 | 0.63 | 0.03 | 0.53 | 2.49 | 0.93 | 1.75 |
| 4 | 60 | P3–P4 | PHASE | 22.27 | 16.2 | 22.23 | 24.77 | 17.8 | 21.95 | 1.43 | 17.77 | 0.73 | 0.65 | 0.91 | 1.25 |
| 4 | 60 | P3/P4 | ASYM | 0.62 | 0.71 | 1.01 | 0.92 | 0.82 | 1.01 | 0.67 | 0.97 | 0.71 | 0.77 | 0.87 | 1.23 |
| 5 | 60 | T3 | MEAN | 5.95 | 4.88 | 6.88 | 4.47 | 6.77 | 6.96 | 7.77 | 3.41 | 0.71 | 1.09 | 0.72 | 1.02 |
| 5 | 60 | T3 | MEANF | 3.35 | 5.88 | 12.24 | 6.43 | 13.5 | 18.99 | 4.4 | 5.18 | 0.48 | 0.91 | 0.44 | 0.91 |
| 5 | 60 | T3 | STDDEV | 3.24 | 1.99 | 3.18 | 2.07 | 2.75 | 2.61 | 1.21 | 1.34 | 0.62 | 0.96 | 0.72 | 1.16 |
| 5 | 60 | T3 | MODFRQ | 1.49 | 5.26 | 9.93 | 13.37 | 17.5 | 24.5 | 39.65 | 32.37 | 0.53 | 0.39 | 0.3 | 0.57 |
| 5 | 60 | T4 | MEAN | 8.2 | 7.01 | 8.56 | 5.05 | 8.92 | 5.91 | 12.22 | 3.05 | 0.82 | 1.39 | 0.79 | 0.96 |
| 5 | 60 | T4 | MEANF | 4.09 | 7.82 | 12.79 | 5.5 | 14.33 | 12.47 | 4.28 | 3.52 | 0.61 | 1.42 | 0.55 | 0.89 |
| 5 | 60 | T4 | STDDEV | 3.79 | 2.86 | 3.22 | 1.99 | 2.57 | 1.87 | 1.07 | 0.85 | 0.89 | 1.44 | 1.11 | 1.25 |
| 5 | 60 | T4 | MODFRQ | 1.77 | 5.24 | 9.87 | 13.32 | 17.56 | 24.28 | 39.6 | 32.36 | 0.53 | 0.39 | 0.3 | 0.56 |
| 5 | 60 | T3–T4 | COHE | 32.02 | 11.33 | 25.7 | 1.92 | 22.12 | 4.98 | 0.7 | 0.03 | 0.44 | 5.9 | 0.51 | 1.16 |
| 5 | 60 | T3–T4 | PHASE | 45.2 | 36.68 | 46.17 | 38.87 | 25.38 | 37.42 | 1.93 | 29.63 | 0.79 | 0.94 | 1.45 | 1.82 |
| 5 | 60 | T3/T4 | ASYM | 0.73 | 0.7 | 0.8 | 0.89 | 0.76 | 1.18 | 0.64 | 1.12 | 0.87 | 0.79 | 0.92 | 1.06 |
| 6 | 60 | O1 | MEAN | 5.12 | 4.39 | 7.08 | 4.26 | 4.09 | 5.14 | 1.98 | 2.68 | 0.62 | 1.03 | 1.07 | 1.73 |
| 6 | 60 | O1 | MEANF | 5.4 | 8.81 | 18.41 | 8.97 | 10.4 | 18.32 | 2.34 | 5.31 | 0.48 | 0.98 | 0.85 | 1.77 |
| 6 | 60 | O1 | STDDEV | 2.59 | 2.74 | 3.32 | 1.92 | 1.38 | 1.44 | 0.73 | 0.9 | 0.83 | 1.43 | 1.99 | 2.41 |
| 6 | 60 | O1 | MODFRQ | 1.57 | 5.24 | 10.02 | 13.24 | 17.32 | 24.65 | 39.85 | 32.27 | 0.52 | 0.4 | 0.3 | 0.58 |
| 6 | 60 | O2 | MEAN | 5.35 | 4.88 | 7.52 | 4.11 | 4.16 | 4.84 | 1.76 | 2.44 | 0.65 | 1.19 | 1.17 | 1.81 |
| 6 | 60 | O2 | MEANF | 5.9 | 9.79 | 20.45 | 8.75 | 10.34 | 16.86 | 2 | 4.46 | 0.48 | 1.12 | 0.95 | 1.98 |
| 6 | 60 | O2 | STDDEV | 2.57 | 3.01 | 3.39 | 1.64 | 1.54 | 1.65 | 0.65 | 0.74 | 0.89 | 1.83 | 1.95 | 2.2 |
| 6 | 60 | O2 | MODFRQ | 1.52 | 5.24 | 10.08 | 13.13 | 17.37 | 24.49 | 39.81 | 32.25 | 0.52 | 0.4 | 0.3 | 0.58 |
| 6 | 60 | O1–O2 | COHE | 27.73 | 14 | 30.62 | 1.5 | 2.73 | 2.38 | 0 | 0.13 | 0.46 | 9.33 | 5.13 | 11.22 |
| 6 | 60 | O1–O2 | PHASE | 14.63 | 15.42 | 12.88 | 16.73 | 13.8 | 16.67 | 19.13 | 16.58 | 1.2 | 0.92 | 1.12 | 0.93 |
| 6 | 60 | O1/O2 | ASYM | 0.96 | 0.9 | 0.94 | 1.04 | 0.98 | 1.06 | 1.12 | 1.1 | 0.96 | 0.87 | 0.91 | 0.96 |

It will be appreciated by persons skilled in the art that numerous variations and/or modifications may be made to the invention as described in the specific embodiments without departing from the spirit and scope of the invention as broadly described. The present embodiments are, therefore, to be considered in all respects as illustrative and not restrictive. Other features and aspects of this invention will be appreciated by those skilled in the art upon reading and comprehending this disclosure. Such features, aspects, and expected variations and modification of the reported results and examples are clearly within the scope of the invention where the invention is limited solely by the scope of the following claims.

We claim:

1. A biofeedback system for administration of electroencephalographic (EEG) neurofeedback training, the system comprising:
a plurality of electrodes sensors for placement on the head of a trainee;
a switching head box comprising a plurality of contacts located at a plurality of contact positions, each of the plurality of contacts being connected to one of the plurality of electrode sensors;
an interface device connected to the switching head box, the interface device comprising at least two EEG signal amplifiers;
a computer comprising software for generating user-control functions which corresponds in real-time to EEG signals received by the interface device and processed by the computer; and
wherein the switching head box comprises a switch comprising a first conductor at a first position which connects a first electrode sensor to a first EEG signal amplifier of the interface device, and a second conductor at a second position which connects a second electrode sensor to a second EEG signal amplifier, for transmitting EEG signals from the trainee to the computer.

2. The biofeedback system of claim 1, wherein the first conductor at a first position of the switch connects only one of the plurality of electrode sensors to the first EEG signal amplifiers, and the second conductor at a second position of the switch connects only one of the plurality of electrode sensors to the second EEG signal amplifier.

3. The biofeedback system of claim 2, wherein the biofeedback system comprises up to 12 electrodes and the switch is movable to up to six contact positions.

4. The biofeedback system of claim 2, wherein the biofeedback system comprises up to 20 electrodes and the switch is movable to up to five contact positions.

5. The biofeedback system of claim 1, wherein the first conductor of the switch connects at least two of the plurality of electrode sensors in parallel to the first EEG signal amplifier, and the second conductor at the second position of the switch connects at least two of the plurality of electrode sensors in parallel to the second EEG signal amplifier.

6. The biofeedback system of claim 1, wherein the number of electrode sensors ranges from 2 to 256.

7. The biofeedback system of claim 1, wherein the number for electrode sensors ranges from 2 to 20.

8. The biofeedback system of claim 1, wherein the number for electrode sensors ranges from 2 to 12.

9. The biofeedback system of claim 1, wherein the switch of the switching head box comprises from up to 10 conductors which connect the plurality of electrode sensors to the interface device.

10. The biofeedback system of claim 1, wherein the interface device comprises from up to 10 EEG signal amplifiers.

11. The biofeedback system of claim 1, wherein the switch further comprises a third conductor which connects a third electrode sensor to a third EEG signal amplifier of the interface device, and a fourth conductor which connects a fourth electrode sensor to a fourth EEG signal amplifier, for transmitting EEG data from the trainee to the computer.

12. The biofeedback system of claim 5, wherein the switching head box further comprises a third conductor which connects at least one electrode sensor to a third EEG signal amplifier of the interface device, and a fourth conductor which connects at least one electrode sensor to a fourth EEG signal amplifier, for transmitting EEG data from the trainee to the computer.

13. A switching head box for use in a system to administer biofeedback training, comprising:
a plurality of electrical contacts at a plurality of contact positions for connection to electrodes sensors on the head of a trainee undergoing biofeedback training;
at least two channel ports (or data lines?);
a switch comprising at least two conductors movable to the plurality of electrical contacts at the plurality of contact positions to connect the electrical contacts to the at least two channel ports (data lines).

14. The switching head box of claim 13, wherein the switching head box comprises from 2 to 256 contacts at located at distinct positions.

15. The switching head box of claim 13, wherein each of the at least two conductors contacts only one electrical contact of the plurality of electrical contacts to connect the one electrical contact to one of the at least two channel ports, when the conductors are moved to each contact position of the plurality of contact positions.

16. The switching head box of claim 13, wherein each of the at least two conductors contacts at least two electrical contacts of the plurality of electrical contacts to connect the at least two electrical contacts to one of the at least two channel ports, when the conductors are moved to each contact position of the plurality of contact positions.

17. A program embodied in a computer readable medium, comprising:
logic that simultaneously identifies at least two independent EEG brainwave signals received by at least two electrical sensors placed on a head of a trainee undergoing biofeedback training;
logic that executes processing of the EEG brainwave signals and records EEG brainwave data derived from the EEG brainwave signals; and
logic that detects a predetermined time setting for processing the EEG brainwave signals and executes a prompt, at the conclusion of the predetermined time setting, to advance a switch if additional electrical sensors are to be processed.

18. The program embodied in a computer readable medium of 17, wherein the program further comprises logic to process a response and to execute processing and recording of EEG brainwave signals of the additional electrical sensors if a response the prompt indicates additional electrical sensors are to be read.

19. The program embodied in a computer readable medium of 17, wherein the program further comprises logic to process a response to the prompt and to execute calculations using the EEG brainwave data if the response to the prompt indicates no additional electrical sensors.

20. The program embodied in a computer readable medium of 17, wherein the program further comprises EEG data files and the program further comprises logic to sort the recorded EEG brainwave data to the appropriate EEG data files.

21. The program embodied in a computer readable medium of 17, wherein the program further comprises logic to execute a prompt to process and display graphic images of the EEG brainwave data.

22. The program embodied in a computer readable medium of 19, wherein the program further comprises logic to execute a prompt, after a response is made which indicates there are no additional electrical sensors, to process and display graphic images of the EEG brainwave data.
